(19) United States
(12) Patent Application Publication
(10) Pub. No.: US 2022/0125972 A1
(43) Pub. Date: Apr. 28, 2022

(54) PROSTHETIC ACCESSORY STERILIZER

(71) Applicant: Johann Cornelius Visser, Woodstock, Cape Town (ZA)

(72) Inventor: Johann Cornelius Visser, Woodstock, Cape Town (ZA)

(21) Appl. No.: 17/438,293

(22) PCT Filed: Mar. 12, 2020
(86) PCT No.: PCT/IB2020/052209

§ 371 Date: Sep. 10, 2021

(30) Foreign Application Priority Data
Mar. 12, 2019 (ZA) 2019/01518

(51) Int. Cl. A61L 2/20; A61L 2/22; A61L 2/26; A61L 9/01; A61L 2/10

(52) U.S. Cl. CPC A61L 2/202; A61L 2/22; A61L 2/26; A61L 9/01; A61L 2/10; A61L 2202/15; A61L 2209/21; A61L 2202/21; A61L 2202/121; A61L 2209/14; A61L 2202/11; A61L 2209/133

(57) ABSTRACT

This invention relates to a prosthetic accessory sterilizer (1), and more particularly, but not exclusively, to a prosthetic liner or sleeve sterilizer. In accordance with this invention there is provided a prosthetic accessory sterilizer comprising an elongate support (3) for receiving a sleeve thereover, a number of ventilation openings (4) in the elongate support (3), air movement means to move air through the ventilation openings (4) and an airborne produced particle that kills bacteria, for example ozone, but not exclusively ozone.

### FIELD OF THE INVENTION

This invention relates to a prosthetic accessory sterilizer, and more particularly, but not exclusively, to a sterilizer for a prosthetic liner or sleeve.

### BACKGROUND TO THE INVENTION

A liner or sleeve (hereinafter a “sleeve”) is used to cover an area of a residual limb over which a prosthesis fits.

Bacteria, sweat, dead skin cells and other contaminants build up in the sleeve from use. Microlesions of the skin may occur and/or be caused by prolonged wear of a prosthesis. This may result in infection.

Infection prevents the user from wearing the prosthesis until the wound heals. This has an effect on the user's general well-being including mobility, daily life, responsibilities and self-confidence.

In order to prevent malodour and infection, the sleeve must be cleaned daily, after use, and especially, after exercise.

Various sterilization methods are available, including the use of alcohol and UV light. The use of alcohol and UV light may have an adverse effect on the lifespan of the sleeve.

US patent application publication number US2015/0064063A1 entitled “Apparatuses and methods for cleaning and drying prosthetic liners” (the “'063 application”) discloses apparatuses that eliminate microbes in a prosthetic liner. These apparatuses eliminate odours and moisture from a prosthetic liner. Apparatuses and methods disclosed illuminate prosthetic liners with anti-microbial lights and push air past the prosthetic liners.

The '063 application discloses air movement means for blowing air into a bottom open end of a prosthetic sleeve. These sleeves are, in most cases, closed off at an opposite end to form a sock. Air pushed into the open end will thus not always circulate efficiently or to a desired extent inside the sleeve. Whilst the apparatus disclosed in the '063 application includes and UV or blue light, it does not disclose an UV or blue light that denatures ozone. The apparatus disclosed does also not include an aromatic substance or a system for applying an aromatic substance to at least part of a sleeve. The '063 does also not disclose the use of an ozone.

U.S. Pat. No. 9,585,498B1 entitled “Prosthetic sleeve holding apparatus” (the “'498 patent”) discloses a prosthetic sleeve holding apparatus. The holding apparatus holds at least one prosthetic sleeve and includes a base member including opposed top and bottom surfaces having a generally planar configuration. The base member includes a first guide ring positioned on the top surface. A support member is rotatably coupled to the base member, the support member having upper and lower surfaces and a circular configuration for rotation about an imaginary vertical axis of rotation. The support member includes a second guide ring situated on the lower surface of the support member. A pair of spaced apart support rods extends upwardly from the support member, each support rod having a cylindrical configuration and a free end displaced from the base member and that is configured to support the prosthetic sleeve.

The apparatus disclosed in the '498 patent does not disclose air movement means for moving air about the inside or outside of a sleeve. Instead, it has ventilation holes in support rods (cylinders) for drawing air from a sleeve draped over one of the rods towards a dehumidifier inside of or being part of the rod. The dehumidifier includes silica packs or other moisture absorbent material. The direction of air movement is from the inside of the sleeve towards the dehumidifier material inside of, or part of the support rod, over which the sleeve is draped. The apparatus of the '498 patent does not include an aromatic dispenser or aromatic dispenser system. It also does not include anti-microbial lights (UV or blue lights) The apparatus also does disclose the use of an ozone or the use of UV light to denature ozone.

### OBJECT OF THE INVENTION

It is an object of this invention to provide a prosthetic accessory sterilizer which, at least partially, alleviates some of the above mentioned difficulties.

### SUMMARY OF THE INVENTION

In accordance with this invention there is provided a prosthetic accessory sterilizer comprising an elongate support for receiving a sleeve thereover, a number of ventilation openings in the elongate support and air movement means to move air through the ventilation openings.

The elongate support extends operatively upright from a base and a cover to locate over the support and a lower open end of the cover to seat in a formation on the base.

A further feature of the invention provides for the prosthetic accessory sterilizer to include an oil diffuser.

There is provided for the diffuser to diffuse oil from an oil reservoir and for the diffused oil to be forced through the elongate support and out of the ventilation openings in the elongate support towards an inside surface of the sleeve.

A further feature of the invention provides for the prosthetic accessory sterilizer to include an ozone source.

There is still further provided for the elongate support to terminate in a thickened end to support part of an inside of the sleeve away from the support.

There is provided for the thickened end to have protrusions extending therefrom and to terminate in rounded ends to space the other part of the sleeve from the thickened end.

The thickened end has a larger cross-section than a cross-section of the support.

A further feature of the invention provides for the cover to include a lid at a top open end thereof.

There is further provided for the cover or the base to have a closeable moisture escape opening.

There is also provided for the sterilizer to include a UV light.

The UV light is included in the base to denature ozone that circulates through the base.

A still further feature of the invention provides for an air flow path to extend from outside the base through filter openings to inside the base into an operatively lower end of the elongate support, operatively upwards in the elongate support and out through ventilation openings in the elongate support and through ventilation openings in the thickened end at the upper end of the elongate support.

The base houses the oil diffuser, electronic components, moisture escape opening, air movement means, UV light or blue light and the ozone source.

There is provided for the oil reservoir to be a tray slidingly removable from the base for refilling thereof.

This invention extends to a method of sterilising a prosthetic accessory comprising the steps of:
placing the accessory over an elongate support;
forcing ozone through the elongate support, out through the ventilation openings in the wall of the elongate support and through the accessory; and
forcing air containing diffused or atomised oil through the elongate support, out through ventilation openings in the wall of the elongate support and through the accessory.

The step of forcing air includes the further step of forcing ozone together with the air through the elongate support.

These and other features of invention are described in more detail below.

### BRIEF DESCRIPTION OF THE DRAWINGS

One embodiment of the invention is described below, by way of example only, and with reference to the drawings in which:

FIG. 1 shows a front perspective view of the prosthetic accessory sterilizer;

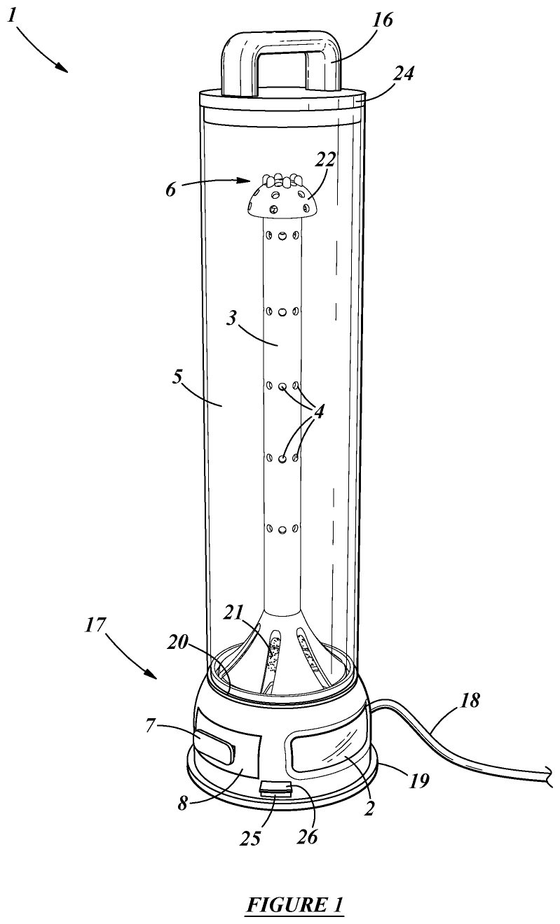

FIG. 2 shows a side view of part of the prosthetic accessory sterilizer, a lid of a cover being excluded from this view and an oil tray removed from the prosthetic accessory sterilizer;

FIG. 3 shows a sectional side view of the prosthetic accessory sterilizer with the lid of the cover included and the tray located in its operative position;

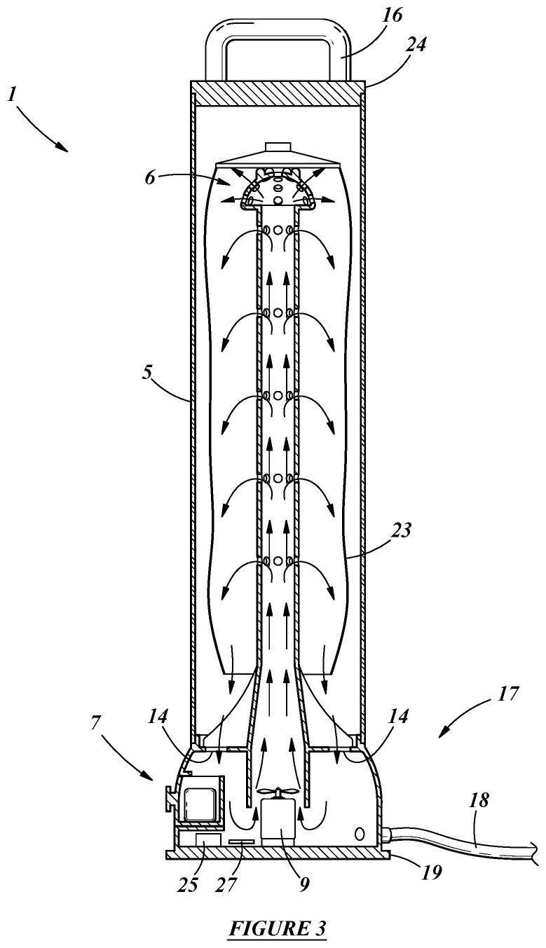

FIG. 4 shows the prosthetic accessory sterilizer with a sleeve in transparent view for illustration purposes drawn over its elongate support;

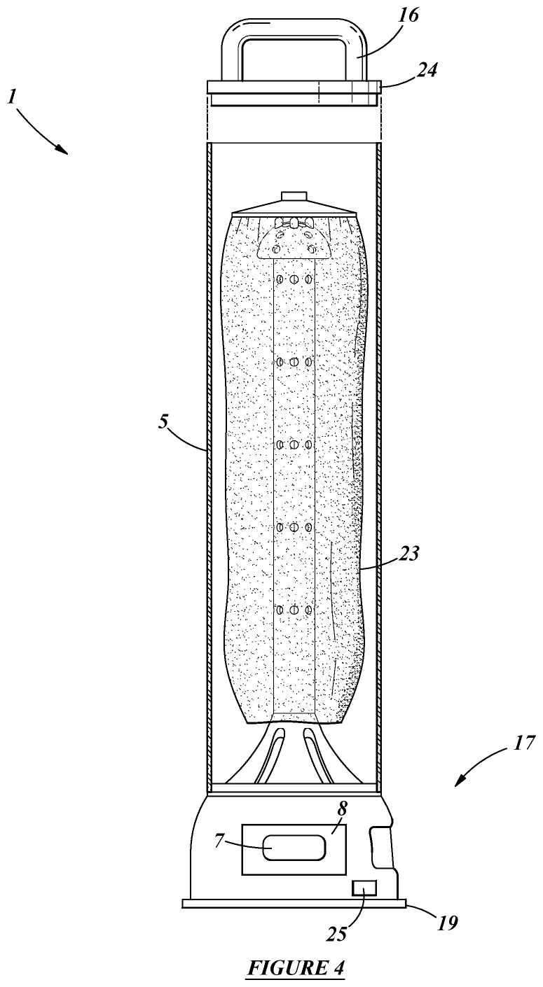

FIG. 5 shows the same view of FIG. 1 of the prosthetic accessory sterilizer but without the lid on the cover; and

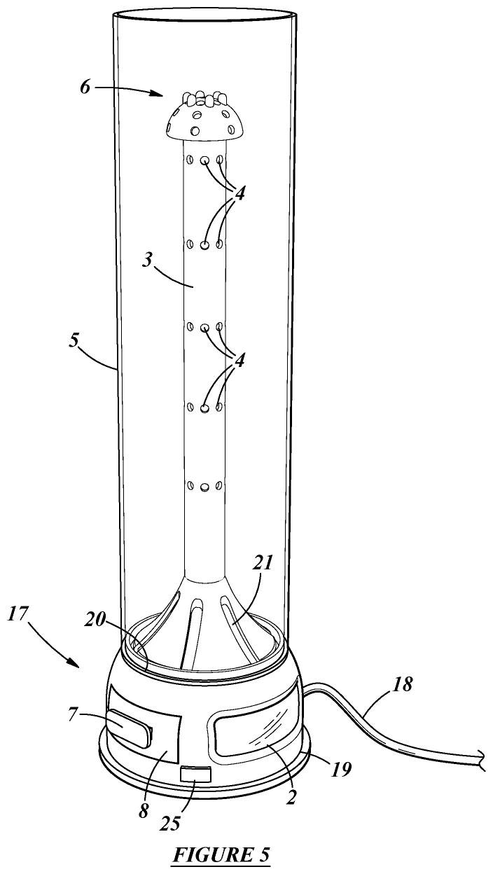

FIG. 6 shows a perspective front view of the lid.

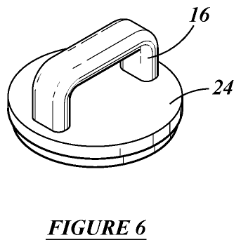

### DETAILED DESCRIPTION OF THE DRAWING

With reference to the drawings, a prosthetic accessory sterilizer for sterilising a prosthetic accessory is generally indicated by reference numeral 1.

The prosthetic sterilizer includes a hollow elongate support 3 for supporting a sleeve 23 thereover. Ventilation openings 4 allow air to flow from inside the support 3 towards an inner surface of the sleeve 23. Air movement means in the form of a fan 9 forces air up into the support 3 and out through the ventilation openings 4.

The accessory sterilizer 1 further includes a base section 17, an electrical supply cable 18, a removable cover 5 and lid 24.

The base 17 has a base plate 19, a top part 20 and a ventilation cavity 21.

The elongate support 3 is a tube which extends operatively upwardly from the centre of the base 17. The elongate support 3 is hollow and cylindrical in shape, having a bottom open end.

The base plate 19 is a disc-shaped bottom section or disc at the operatively bottom of the base 17.

The top part 20 extends from the base plate 19. The top part 20 is cylindrically or substantially dome-shaped and forms a lower housing as described further below. The apex of the dome 20 supports the elongate support 3 therein. A bottom end of the elongate support 3 terminates inside the dome 20.

An oil tray 7 is situated in a side of the dome 20. The oil tray 7 is and open-ended box, open at its operatively top. An outside surface of an outer wall of the oil tray 7 is complementary curved to the surface of the dome 20 to provide support walls (side walls and a bottom wall) for the tray. A handle extends from the outside surface or wall of the tray 7. The handle is rectangular with curved corners.

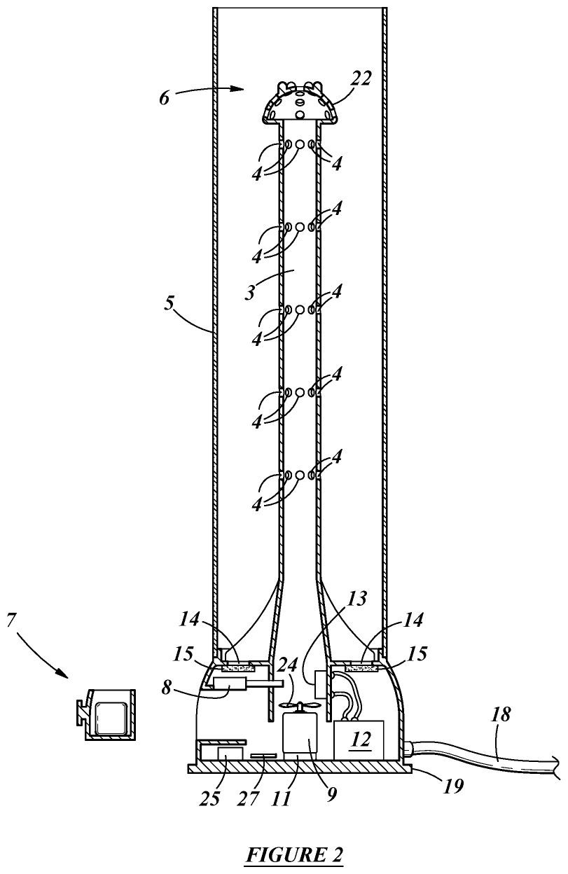

The support walls, complementary to a bottom and side surfaces of the tray 7, extend inwards from the dome 19, as shown in FIG. 2.

An air vent opening 25 is located on a side of the dome 20. The air vent opening 25 is rectangular in shape and extends through the side wall of the dome 20. A door 26 covers the opening. The door 26 is electronically operable and is moveable between a closed and opened position.

A control panel 2 is situated on or in a side of the dome 20. The control panel 2 is substantially rectangular in shape. The electronic control panel 2 controls the functionality of the components within the dome 20. The control panel 2 includes a timer (not shown) to control electronics of the components and thus the operation of the sterilizer 1. The timer allows for the electronic components to be switched on and/or off after a certain period of time or at a scheduled time.

A LED light 27, which emits a UV light such as a UVB light, is located on an operatively inside surface of the base plate 19.

A power cable 18 extends from the dome 20.

A flat operatively horizontal, ring-shaped divider wall, divides the base in a bottom housing and upper apex or frusto conical section. The wall extends around the elongate support up to a circumferential rim on the outside of the domed base. The outer circumference the rim forms an outer flat ring or step or seat around the base of the apex for receiving a bottom edge of the cover 5.

Ventilation openings 14 are spaced apart around the elongate support on the flat ring-shaped surface of the dome 20.

As shown in FIG. 2, air filters 15 are attached to the underside (inside) surface of the divider wall, below and covering ventilation openings 14 in the divider wall.

A diffuser 8 is attached to the inside of the bottom housing of the dome 20 at a top end of a tray cavity. The diffuser 8 has a nozzle that extends through a bottom end of the support 3 and terminates inside the support 3, above a fan 9.

The fan 9 is centrally located in the base 17. The fan impeller 24 is attached to a motor 9 attached to the base plate 19. The impeller 24 is situated at a top end of the motor 9.

The bottom open end of the elongate support 3 extends over the fan 9 with a bottom edge, just below its impeller 24.

An ozone generator 13 is situated on an inside surface at a lower end of the elongate support 3, above the fan impeller 24.

A ventilation cavity 21 is formed between and upside down (frusto conical) funnel or apex section with slots therein and the bottom end of the elongate support 3. The slots correspond with the filter openings 14 in the flat ring-shaped wall of the dome 20.

The elongate support 3, in the form of a tube, extends upwards from the dome 20 and terminates in a thickened end 22. The thickened end 22 is dome-shaped and has a larger diameter than the tube. The elongate support 3 has a larger diameter inside the dome 20. Thereafter the elongate support 3 tapers to a smaller diameter to where it joins with the apex of the slotted-funnel 21. The rest of the elongate support 3 is right-circular up to the thickened end 22.

Ventilation openings 4 are spaced apart on and about the wall of the elongate support tube 3.

Stubs 6 extend from the top surface of the thickened end 22. The stubs 6 are rounded, concave at their tops.

The cylindrical hollow cover 5, when in position, seats on the dome 20 in the step described above and covers the elongate support 3. The top end of the cover 5 is open.

A lid 24 seats on the top open end of the cover 5. A handle 16 extends from the lid 24. The lid 24 has a seal around a bottom edge thereof to form an air-tight seal between the lid 24 and cover 5.

An air flow path extends from outside the base through ventilation openings 14, or air vent opening 25, to inside the base 17 into an operatively lower end of the elongate support 3, operatively upwards in the elongate support 3 and out through ventilation openings 4 in the elongate support 3 and through ventilation openings 4 in the thickened end 22 at the upper end of the elongate support 3.

In use, a prosthesis sleeve 23 is placed over the elongate support 3. The stubs 6 space the prosthesis sleeve 23 from an outer surface of the thickened end 22 in order to improve air distribution. The thickened end 22 also ensures that the sleeve 23 hangs away from the tube 3 i.e. spaced therefrom. The cover 5 is placed on the base 17. The lid 24 is located on the cover 5 using the handle to place the lid 24. The electrical cable 18 is connected to an electrical source. The fan 9 causes air circulation within the sterilizer 1, as described below. At this time, both the diffuser 8 and the ozone generator 12 may be operative or either one may be selectively operative. The control panel 2 may be used to selectively activate or de-activate the diffuser 8 and/or ozone generator 12. Oil is placed inside the oil tray 7. The diffuser 8 transports the oil to, or diffuses the oil, over the fan 9. Air enters the funnel 21 and dome 20 through the filter openings 14 in the funnel 21 and dome 20. The air is filtered through the filters 15. The fan 9 pushes the air upwards inside the elongate support 3 and through the ventilation openings 4. The ozone generator 12 and the diffuser 8 causes the air to be mixed with diffused oil and/or ozone before the air is pushed to and through the ventilation openings 4 in the elongate support 3. The oil impregnated air and ozone will push through the ventilation openings 4 and onto an inner surface of the sleeve 23 in case of an air impervious sleeve 23. In case of an air-pervious sleeve 23 some of the air diffused oil and/or ozone will travel through the sleeve 23. It is possible for the user to require that the ozone need to be denatured at a faster rate, therefore an UVB light 27 could be switched on at the control panel 2 for any desired period of time. The UVB light 27 removes ozone at an accelerated pace. In any event, whether or not the air or part thereof flows through the sleeve 23, the cover 5 and lid 24 contain and circulate the air inside the sterilizer 1. In order for the ozone to work optimally, the door 26 of the air vent opening 25 will be opened to first release any excess moisture in the air inside the cover 5. Thereafter the door 26 will be closed to keep the ozone within the cover 5.

It is envisaged that the sterilizer 1 described above herein will be convenient to use to sterilise a prosthetic sleeve with ozone and to diffuse oil for aromatic effect. In some cases the oil may include anti-microbial substances which will be diffused and circulated as with the oil. Ozone is emissive and should also not be inhaled and although the cover 5 and lid 24 might not provide an airtight seal the cover 5 and lid 24 mostly contain the ozone inside the sterilizer 1. The UV light also denatures the ozone. The lid 24 makes it easy for a user to place the sleeve or liner 23 inside the sterilizer 1.

It will be appreciated by those skilled in the art that many other embodiments are possible without departing from the scope of the invention. For example, the prosthetic accessory sterilizer may have either a diffuser or an ozone generator or both. The cover may also be airtight or it may be adjusted to allow for a small passage for small volumes of air to enter the sterilizer. The control may be located remotely from the sterilizer, the sterilizer may even be remote controlled. The housing or dome may be any convenient shape. The elongate support may be any substantially elongate, hollow column and need not be circular with different diameters as disclosed.

1. A prosthetic accessory sterilizer comprising an elongate support for receiving a sleeve thereover, a number of ventilation openings in the elongate support and air movement means to move air through the ventilation openings.

2. The prosthetic accessory sterilizer as claimed in claim 1 in which the elongate support extends operatively upright from a base and has a cover locatable over the support and a lower open end of the cover to seat in a seat formation on the base.

3. The prosthetic accessory sterilizer as claimed in claim 1 in which the prosthetic accessory sterilizer includes an oil diffuser.

4. The prosthetic accessory sterilizer as claimed in claim 3 in which the diffuser diffuses oil from an oil reservoir.

5. The prosthetic accessory sterilizer as claimed in claim 4 in which the diffused oil is forced through the elongate support and out of the ventilation openings in the elongate support towards an inside surface of the sleeve.

6. The prosthetic accessory sterilizer as claimed in claim 1 in which the prosthetic accessory sterilizer includes an ozone source.

7. The prosthetic accessory sterilizer as claimed in claim 1 in which the elongate support terminates in a thickened end to support part of an inside of the sleeve thereon.

8. The prosthetic accessory sterilizer as claimed in claim 7 in which the thickened end has protrusions extending therefrom to support the sleeve thereon, spaced away from the thickened end.

9. The prosthetic accessory sterilizer as claimed in claim 8 in which the protrusions terminate in rounded ends.

10. The prosthetic accessory sterilizer as claimed in claim 7 in which the thickened end has a larger cross-section than a cross-section of the support so that the sleeve is spaced from the support when it is draped over the thickened end.

11. The prosthetic accessory sterilizer as claimed in claim 2 in which the cover includes a lid at a top open end thereof.

12. The prosthetic accessory sterilizer as claimed in claim 2 in which the cover or the base has a closeable moisture escape opening.

13. (canceled)

14. The prosthetic accessory sterilizer as claimed in claim 1 in which sterilizer includes an UV light.

15. (canceled)

16. The prosthetic sterilizer as claimed in claim 1 in which an air flow path extends from outside the base through filter openings in the base into an operatively lower end of the elongate support, operatively upwards in the elongate support and out through ventilation openings in the elongate support and through ventilation openings in the thickened end at the upper end of the elongate support.

17. The prosthetic accessory sterilizer as claimed in claim 1 in which the base houses the oil diffuser, electronic components, a moisture escape opening, air movement means, UV light or blue light and/or an ozone source.

18. The prosthetic accessory sterilizer as claimed in claim 1 in which the elongate support includes a series of ventilation openings.

19. The prosthetic accessory sterilizer as claimed in claim 4 in which the oil reservoir is a tray slidingly removable from the base for refilling thereof.

20. A method of sterilising a prosthetic accessory comprising the steps of:
placing the accessory over an elongate support; and
moving air from within the support, with air movement means, through a number of ventilation openings in the elongate support towards the accessory.

21. The method as claimed in claim 20 further including the steps of:
generating ozone to move ozone rich air through the elongate support, out through ventilation openings towards the accessory.

22. The method of sterilising a prosthetic accessory as claimed in claim 20 including the further step of:
moving air containing diffused or atomised oil through the elongate support, out through the ventilation openings in the wall of the elongate support and towards the accessory.
